(19) United States
(12) Patent Application Publication
(10) Pub. No.: US 2006/0052312 A1
(43) Pub. Date: Mar. 9, 2006

(54) Paclitaxel hybrid derivatives

(76) Inventors: Paul W. Erhardt, Sylvania, OH (US); Weislaw A. Klis, The Woodlands, TX (US); Jeffrey G. Sarver, Rossford, OH (US)

Correspondence Address: MACMILLAN SOBANSKI & TODD, LLC;ONE MARITIME PLAZA FOURTH FLOOR, 720 WATER STREET, TOLEDO, OH, 43604-1619, US

(21) Appl. No.: 11/221,391

(22) Filed: Sep. 6, 2005

Related U.S. Application Data
(63) Continuation of application No. PCT/US04/07269, filed on Mar. 5, 2004.
(60) Provisional application No. 60/452,649, filed on Mar. 7, 2003.

(51) Int. Cl. A61K 38/06; A61K 38/05; A61K 38/04; A61K 31/337

(52) U.S. Cl. 514/18; 514/19; 514/449

(57) ABSTRACT

Methods and compositions for treating cancer patients that include administering at least one or more hybrid derivatives of paclitaxel that simultaneously display improved aqueous solubility, chemical stability under physiological conditions, a decreased liability toward multi-drug resistance, and in certain instances enhanced selective toxicity toward cancer cells compared to normal cells. The derivative, paclitaxel substituted with at least one or more polar appendages at either the 7- or 10-positions as defined by a formula “7-OR-10-OR′-paclitaxel”, is either deployed alone or in combination protocols with other chemotherapeutic agents.

### BACKGROUND OF THE INVENTION

Paclitaxel (PAC) is a chemotherapeutic agent that is given by injection to treat various forms of cancer, particularly breast cancer. Although PAC is regarded as a very effective drug, there are three areas in which its overall clinical profile would benefit from further improvements. First, PAC's low aqueous solubility has necessitated that its formulations also contain undersirably high levels of solubility enhancing agents. Second, PAC is readily subject to multidrug resitance (MDR) whereupon its chemotherapeutic efficacy becomes significantly attenuated when cancers begin to exhibit the MDR phenomena. Finally, like many other anticancer agents whose beneficial effects are derived from an interruption of the cell division process, PAC does not exhibit a high degree of selectivity for cancer cells versus healthy cells in the body that are also undergoing rapid cell division.

While the synthetic approaches toward the general chemical arrangements have been published by the inventors here (Klis, W. A., Sarver, J. G., Erhardt, P. W., Mechanistic Considerations Pertaining To The Solvolysis Of Paclitaxel Analogs Bearing Ester Groups At The C2′ Position, Tetrahedron Letters, 2001, 42: 7747-7750; Klis, W., Sarver, J., and Erhardt, P., Selective conversion of 2′,7-Bis-Monochloroacetylpaclitaxel Analogs to 7-Monochloroacetyl Derivatives by Solvolysis in Methanol, Synthetic Communications, 2002, 32, 2711-2718) and are the subject of a pending U.S. Patent (Erhardt, P., Klis, W. and Sarver, J., Selective Conversion of 2′7-Bis-Monochloroacetyl-paclitaxel Analogs to 7-Monochloroacetyl Derivatives by Solvolysis in Methanol, PCT/US02/30727 which claims priority to U.S. Ser. No. 60/327,406 filed Oct. 5, 2001), the specific compounds that represent the preferred embodiments of the hybrid derivatives and their specific synthesis of the present invention have not yet been published or disclosed.

### SUMMARY OF THE INVENTION

In one aspect the present invention relates to a method for treating cancer patients by administering hybrid derivatives of paclitaxel that simultaneously display improved aqueous solubility, chemical stability under physiological conditions, and a decreased liability toward multi-drug resistance. The derivatives are deployed alone or in combination protocols with other chemotherapeutic agents.

In certain embodiments, the hybrid derivatives contain appendages attached to the 7-position of paclitaxel, the 7-position of 10-deacetylpaclitaxel, the 10-position of 10-deacetylpaclitaxel, or the 10-position of 7-acyl-10-deacetylpaclitaxel where the acyl group includes but is not limited to acetyl, chloroacetyl and methoxyacetyl.

In certain embodiments, the attachments are via ester linkages which use the hydroxy group inherently present at the 7-position or the hydroxy group that becomes exposed at the 10-position after deacyetylation of the paclitaxel. In certain embodiments, the appendages are partially protected amino acids or, alternatively, are completely unprotected amino acids for which either type can be attached via the amino acid's terminal or, when present, side-chain carboxylic acid moieties. The amino acids include but are not limited to [(CH₃)₃COCO]N-Asp, [φCO]N-Asp, Asp[CH(CH₃)₂], Asp[CH₂φ] or Asp wherein the β-carboxylic acid moiety is used to form the ester linkage.

In another aspect of the present invention, the hybrid derivatives additionally display selective toxicity toward cancer cells compared to normal cells. The appendages are adducts that are attached directly via an inherent. carboxylic acid moiety or are adducts further connected to a linker molecule having a carboxylic acid that can serve as the attachment. The linker can be a connecting chain between 1 to 10 carbons that also bears one or more additional chemical functionalities that can increase aqueous solubility. Such functionality includes but is not limited to one or more combinations of an alcohol group, an amino group, or a carboxylic acid group.

The adduct can be a derivative of a small peptide. Where the peptide has two to ten amino acids in either a linear, branched or cyclic arrangement. In certain embodiments the peptide comprises an Asn-Gly-Arg, [acyl]N-Asn-Gly-Arg, Gly-Asn-Gly-Arg-Gly or Cys-Asn-Gly-Arg-Cys-Gly motif that preferentially distributes to the neovascularization of a tumor.

In other embodiments, the peptide comprises:

an Arg-Gly-Asp, [acyl]N-Arg-Gly-Asp, Arg-Gly-Asp-Ser, [acyl]N-Arg-Gly-Asp-Ser or cyclic(-Arg-Gly-Asp-(D)-Phe-[N-Methyl]-Val-) motif that preferentially distributes to integrin receptors over-expressed by cancer cells;

a δ-Glu-δ-Glu-NH₂ motif that associates with the PSMA enzyme produced by prostate cancer cells;

a Glutaryl-Hyp-Ala-Ser-Chg-Gln-Ser-Leu motif that associates with the PSA enzyme produced by prostate cancer cells; or,

a β-Ala-Leu-Ala-Leu or [HO₂C(CH₂)₂CO]N-β-Ala-Leu-Ala-Leu motif that associates with a peptidase enzyme over-expressed by cancer cells.

In certain other embodiments, the adduct comprises:

a derivative of a 1,2,3-trisubstituted β-lactam that inhibits the PSA enzyme produced by prostate cancer cells;

a derivative of a 4,6-disubstituted quinazoline system that associates with the EGFR, HER-2 and ErbB pathways over-expressed within cancer cells;

a derivative of a 5,6,7,8-tetrahydro-1,8-naphthyridin-2-yl system that preferentially distributes to integrin receptors over-expressed by cancer cells;.

a derivative of folic acid that is able to use the folate transporter to enhance its uptake into cancer cells;

a derivative of spermine or of metuporamine C that is able to use the polyamine transporter to enhance its uptake into cancer cells or to decrease metastases by interrupting cancer cell invasion and motility;

a derivative of cholic acid that is able to use the cholate transporter to enhance its uptake into cancer cells;

a derivative of 2-methoxyestradiol or of genistein that associates with estrogen receptors over-expressed by cancer cells;

a derivative of testosterone that associates with androgen receptors over-expressed by cancer cells during early stage prostate cancer; or,

a derivative of ascorbic acid that is able to use the SVCT2 transporter to enhance its passage across the blood-brain barrier so as to treat brain cancers.

In yet another aspect, the present invention relates to the compositions of matter where paclitaxel is substituted with one polar appendage at either the 7- or 10-positions as defined by the formula 7-OR-10-OR′-paclitaxel where

R is an appendage-, acyl- or H—;

R′ is an appendage-, acetyl- or H—; where the appendage is a polar adduct initially having a free carboxy-group so as to directly allow formation of an ester link to paclitaxel or has a hydroxy- or amino- group so that the latter can be attached to a connecting chain that then initially bears a free carboxy-group so as to allow formation of an ester link to paclitaxel. Alternatively, when the appendage is a non-polar adduct, it has a carboxy-, hydroxy- or amino-group so that it can be attached to a polar connecting chain that bears a free carboxy-group so as to allow formation of an ester link to paclitaxel;

acyl is an acetyl-, chloroacetyl- or methoxyacetyl-;

the adduct is a small-peptide derivative having from 2 to 10 amino acid units, small organic molecules having molecular weights less than 500 grams that are derivatives of the following templates: 1,2,3-trisubstituted β-lactam; 4,6-disubstituted quinazoline; 5,6,7,8-tetrahydro-1,8-naphthyridin-2-yl; folic acid; polyamine; metupuramine C; cholic acid; estrogen; phytoestrogen; androgen; or, ascorbic acid; and

the connecting chain is a non-polar alkyl or alkene straight or branched chain having 2 to 10 carbons and two carboxylic acid moieties, polar alkyl or alkene straight or branched chain having 2 to 10 carbons and three or more carboxy-, hydroxy- or amino-groups, or a non-polar or polar small peptide of 1 to 5 amino acids.

In certain embodiments the appendage is:

a polar adduct having the formula [(CH₃)₃COCO]N-Asp, Asp-[CH₂φ], or Asp, all of which are directly linked to paclitaxel by their β-carboxylic acid moiety;

a polar adduct having the formula Asn-Gly-Arg, Gly-Asn-Gly-Arg-Gly, or c[-Cys-Asn-Gly-Arg-Cys-]Gly, all of which are directly linked to paclitaxel by their terminal carboxylic acid moiety;

a polar adduct having the formula Arg,-Gly-Asp, [Acetyl]N-Arg-Gly-Asp, Arg-Gly-Asp-Ser,[Acetyl]N-Arg-Gly-Asp-ser, or c[-Arg-Gly-Asp-(D)-Phe[N-R′″] Val-], all of which are directly linked to paclitaxel by their terminal carboxylic acid moiety except for the cyclized motif which uses R′″═CH₂CH₂CO₂H to form the attachment;

a polar adduct having the formula γ-Glu-γ-Glu-Gly directly linked to paclitaxel by the terminal carboxylic acid moiety;

a polar adduct having the formula [Glutaryl]N-Hyp-Ala-Ser-Chg-Gln-Ser-Leu directly linked to paclitaxel by the terminal carboxylic acid moiety;

a non-polar adduct having the formula β-Ala-Leu-Ala-Leu attached to a polar connecting chain by the terminal carboxylic acid moiety, where the polar connecting chain is then linked to paclitaxel by its own carboxylic acid moiety;

a non-polar adduct having the formula of a 1,2,3-trisubstituted β-lactam system attached to a polar connecting chain by a carboxylic acid function, where the polar connecting chain is then linked to paclitaxel by its own carboxylic acid moiety;

a non-polar adduct having the formula of a 4,6-disubstituted quinazoline system attached to a polar connecting chain by an amino function, where the polar connecting chain is then linked to paclitaxel by its own carboxylic acid moiety;

a polar adduct having the formula of 5,6,7,8-tetrahydro-1,8-naphthyridin-2-yl system whose side chain ends with an Asp directly linked to paclitaxel by the α-carboxylic acid moiety;

a polar adduct having the formula of a folic acid derivative directly linked to paclitaxel by its terminal carboxylic acid moiety;

a polar adduct having the formula of spermine or metuporamine C attached to a non-polar connecting chain by either a central or terminal amino function, where the non-polar connecting chain is then linked to paclitaxel by its own carboxylic acid moiety;

a polar adduct having the formula of cholic acid, taurocholic acid or glycolic acid attached to a non-polar connecting chain by an amido function, where the non-polar connecting chain is then linked to paclitaxel by its own carboxylic acid moiety;

a polar adduct having the formula of a aspartylcholic acid system directly linked to paclitaxel by its β-carboxylic acid moiety;

a non-polar adduct having the formula of a 2-methoxyestradiol derivative attached to a polar connecting chain by an alcohol group, where the polar connecting chain is then linked to paclitaxel by its own carboxylic acid moiety;

a moderately polar adduct having the formula of a genistein derivative attached to a non-polar or polar connecting chain by an alcohol group, where the connecting chain is then linked to paclitaxel by its own carboxylic acid moiety;

a non-polar adduct having the formula of a testosterone derivative attached to a polar connecting chain by an alcohol group, where the connecting chain is then linked to paclitaxel by its own carboxylic acid moiety; or,

a polar adduct having the formula of an ascorbic acid derivative that is attached to a non-polar connecting chain by an alcohol, where the non-polar connecting chain is then linked to paclitaxel by its own carboxylic acid moiety.

### BRIEF DESCRIPTION OF THE DRAWINGS

FIG. 1 shows Paclitaxel: R═COCH₃ and R′═H. Baccatin III: Paclitaxel minus the entire 13-position substituent. Docetaxel: R═R′═H and C₆H₅CONH replaced by (CH₃)₃COCONH.

FIG. 2 shows polar appendages: (a) Acidic moiety (BOC=t-butoxycarbonyl); (b) Basic moiety (Bnz=Benzyl); and (c) Dual acid and base moiety. Using the chemical methods describe herein, these types of appendages can be placed at either R or R′, so as to simultaneously obtain increased aqueous solubility and decreased MDR liability.

FIG. 3 shows examples of cancer selectivity adducts: Amino acid sequences are specified by either one-letter or three-letter codes similar to how each substance is commonly conveyed within the technical literature; Arrows indicate location of attachment to PAC according to the preferred MDR-lowering substitution pattern along PAC's northern edge (an additional linking fragment may also be used as part of the connection); Multiple arrows indicate that more than one option can be deployed for connection (but no more than one connection will be used within a given construct); Groups in brackets behind each arrow indicate functionality removed from parent adduct so as to allow for the connection; Words below each adduct describe the mechanism that affords selectivity for cancer cells over normal cells, generally because the indicated system becomes over-expressed in cancer cells; The numbers in parentheses pertain to references that are complied at the end of this document, all of which are expressly incorporated herein be reference. The acronym after the reference indicates to what types of anti-cancer agents the adduct may already have been attached; most often this has been doxorubicin or ‘DOX’.

Other objects and advantages of the present invention will become apparent to those skilled in the art upon a review of the following detailed description of the preferred embodiments and the accompanying drawings.

### DETAILED DESCRIPTION OF THE PREFERRED EMBODIMENTS

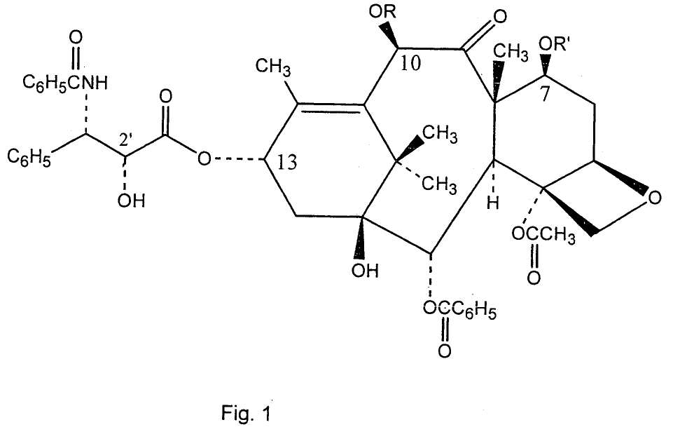

In one aspect, the present invention relates to the structural features in drugs that pertain to interaction with a P-glycoprotein transporter system (Pgp) that is largely responsible for PAC-related MDR. Uniquely distinguishable from all prior art in this area, however, the present invention relates to structural features that reduce Pgp binding so that those features are incorporated into drugs, such as PAC, in order to help such drugs avoid Pgp and the accompanying MDR-related fall-off in their chemotherapeutic efficacies. To attach the MDR-avoiding features onto PAC it was necessary to first identify a neutral region on PAC where the addition of appendages do not alter PAC's inherent anticancer mechanism, namely an over-stabilization of the microtubule system within cells that then interrupts the cell cycle process for which rapidly dividing cells are extremely dependent. The appendages can be placed along the northern edge of PAC without significantly altering its inherent anticancer activity, as shown in FIG. 1. Chemical methods are established that can be used to readily manipulate PAC along its northern edge. The latter requires initial protection of PAC's 2′-position followed by its de-protection subsequent to such manipulations. The 2′-protection and de-protection chemistry was accomplished by the present co-inventors, as disclosed in pending patent application entitled “Selective Conversion of 2′,7-Bis-Monochloracetylpaclitaxel Analogs to 7-Monochloroacetyl Derivatives by Solvolysis in Methanol, PCT/US02/30727, which is expressly incorporated herein by reference along with all other references mentioned herein. In terms of the chemical manipulations needed along the northern edge, the present invention relates to ester connections deployed at the 7- or 10-positions of PAC in that such systems demonstrate remarkable aqueous stability at pH 7.4 and good stability within cell culture assays as shown in Table 1 below. Even more surprising is that some of these simple ester arrangements also reduce PAC's MDR liability from about 1000-fold to about 150-fold.

TABLE 1
Stability and activity of PAC esters (R and R′ refer to FIG. 1).

| | | T½ | Potency | MDR |
|---|---|---|---|---|
| R | R′ | (hrs.)^{a} | (nM)^{b} | Liability^{c} |
| COCH₃ | H^{d} | 278 | 6.9 | 1041 |
| COCH₃ | COCH₂Cl | 597 | 9.3 | 341 |
| COCH₂Cl | COCH₂Cl | 600 | 41.0 | 142 |

^{a}Apparent half-life in aqueous media at pH 7.4 and 37° C..
^{b}Dose that inhibits MCF7 human breast cancer cell growth by 50% (non-MDR cell line.
^{c}Ratio of doses inhibiting growth by 50% in MDR over non-MDR human breast cancer cell lines.
^{d}PAC.

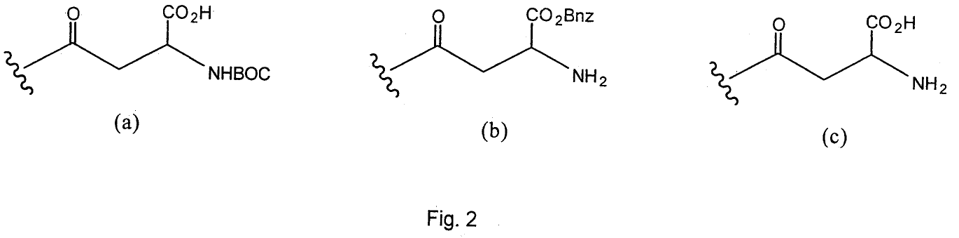

In another aspect of the present invention, highly polar functionalities are included as part of the ester-appended features. Both an acidic and a basic functionality have been added as well as an amino acid containing moiety, as shown in FIG. 2. Although several other investigators have previously explored the northern edge of AC, and especially of baccatin III (FIG. 1), in terms of non-polar appendages (e.g. Alstadt, T. J., et al, Synthesis and Antitumor Activity of Novel C-7 Paclitaxel Ethers: Discovery of BMS-184476, Journal of Medicinal Chemistry, 2001, 44: 4577-4583; Ojima, I., et al., New Taxanes as Highly Efficient Reversal Agents For Multi-Drug Resistance in Cancer Cells, Bioorganic & Medicinal Chemistry Letters, 1998, 8:189-194), very little work has been done using even moderately polar groups (Georg, G. I., Y. Liu, and T. C. Boge, 7-O-Acylpaclitaxel Analogues: Potential Probes to Map the Paclitaxel Binding Site, Bioorganic & Medicinal Chemistry Letters, 1997, 7: 1829-1832; Bhat, L., et al., Synthesis and Evaluation of Paclitaxel C7 Derivatives: Solution Phase Synthesis of Combinatorial Libraries, Bioorganic & Medicinal Chemistry Letters, 1998, 8:3181-3186). Moderately polar functionality has been placed along the northern edge of docetaxel, a closely related drug (FIG. 1), but again, studies in this regard have been very limited (Uoto, K, et al., Synthesis and Evaluation of Water-Solublie Non-Prodrug Analogs of Docetaxel Bearing sec-Aminoethyl Group at the C-10 Position, Chem. Pharm. Bulletin, 1998, 46: 770-776). To the inventors' knowledge, the highly polar features utilized in the present invention have not been previously reported within the published PAC-related literature.

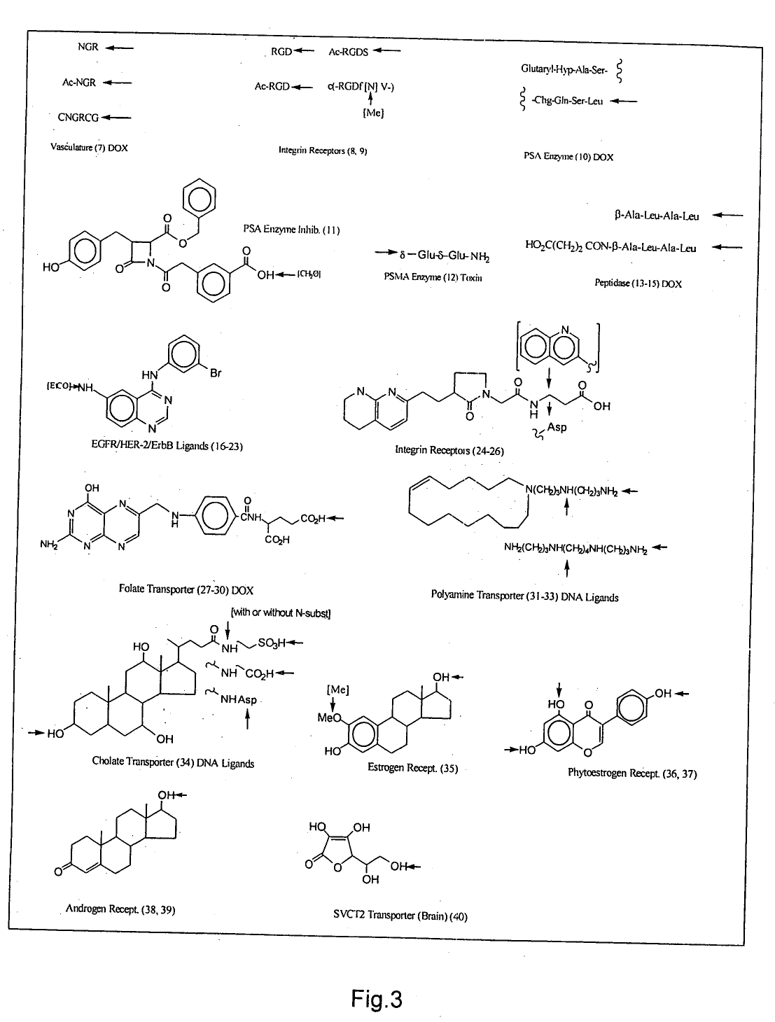

Furthermore, while others have concluded that “modifications at the 7-position of PAC are detrimental to activity” (5), the unexpected and unique attributes of the novel arrangements of the present invention include: (i) increased aqueous solubility making the present analogs very amendable to improved clinical formulations; (ii) significantly decreased liability toward MDR-related reductions in potency; and, (iii) the capability to selectively enhance toxicity toward cancer cells versus healthy, rapidly dividing cells by either utilizing certain polar adducts directly linked to PAC via the ester arrangements with the present invention and/or by further utilizing the acidic, basic or amino acid containing, appendages of the present invention as connecting linkages to various of such polar or non-polar adducts. FIG. 3 provides examples of the adducts that are readily appended to PAC according to the methods of the present invention so as to provide stable AC hybrid derivatives that exhibit all three of the attributes listed above. FIG. 3 also shows the type of pharmacological selectivity that accompanies each adduct along with a reference in that regard, and an arrow indicative of the chemical connection that can be deployed during synthesis based upon the chemical methods of the present invention. The various adducts are either joined to PAC directly according to one preferred MDR-lowering substitution pattern along the northern edge, or they are joined to the polar appendages shown in FIG. 2 according to one preferred MDR-lowering substitution pattern. In both cases, an additional linking fragment may also be incorporated as part of the connection.

The above detailed description of the present invention is given for explanatory purposes. It will be apparent to those skilled in the art that numerous changes and modifications can be made without departing from the scope of the invention. Accordingly, the whole of the foregoing description is to be construed in an illustrative and not a limitative sense, the scope of the invention being defined solely by the appended claims.

### LIST OF REFERENCES

1. Erhardt, P. W., W. A. Klis, and J. G. Sarver, Selective Conversion of 2′,7-Bis-Monochloroacetylpaclitaxel Analogs to 7-Monochloroacetyl Derivatives by Solvolysis in Methanol. 2001: U.S. Ser. No. 09/______.
2. Alstadt, T. J., et al., Synthesis and Antitumor Activity of Novel C-7 Paclitaxel Ethers: Discovery of BMS-184476. Journal of Medicinal Chemistry, 2001. 44: 4577-4583.
3. Ojima, I., et al., New Taxanes as Highly Efficient Reversal Agents For Multi-Drug Resistance in Cancer Cells. Bioorganic & Medicinal Chemistry Letters, 1998. 8: 189-194.
4. Georg, G. I., Y. Liu, and T. C. Boge, 7-O-Acylpaclitaxel Analogues: Potential Probes to Map the Paclitaxel Binding Site. Bioorganic & Medicinal Chemistry Letters, 1997. 7: 1829-1832.
5. Bhat, L., et al., Synthesis and Evaluation of Paclitaxel C7 Derivatives: Solution Phase Synthesis of Combinatorial Libraries. Bioorganic & Medicinal Chemistry Letters, 1998. 8: 3181-3186.
6. Uoto, K., et al., Synthesis and Evaluation of Water-Soluble Non-Prodrug Analogs of Docetaxel Bearing sec-Aminoethyl Group at the C-10 Position. Chem. Pharm. Bulletin, 1998. 46: 770-776.
7. Curnis, F., et al., Differential Binding of Drugs Containing the NGR Motif to CD13 Isoforms in Tumor Vessels, Epithelia, and Myeloid Cells. Cancer Research, 2002. 62: 867-874.
8. Dechantsreiter, M. A., et al., N-Methylated Cyclic RGD Peptides as Highly Active and Selective ανβ₃ Integrin Antagonists. Journal of Medicinal Chemistry, 1999. 42: 3033-3040.
9. Meinecke, R. and B. Meyer, Determination of the Binding Specificity of an Integral Membrane Protein by Saturation Transfer Difference. NMR: RGD Peptide Ligands Binding to Integrin α_{IIb}β₃. Journal of Medicinal Chemistry, 2001. 44: 3059-3065.
10. Garsky, V. M., et al., The Synthesis of a Prodrug of Doxorubicin Designed to Provide Reduced Systemic Toxicity and Greater Target Efficacy. Journal of Medicinal Chemistry, 2001. 44: 4216-4224.
11. Adlington, R. M., et al., Design, Synthesis, and Proposed Active Site Binding Analysis of Monocyclic 2-Azetidinone Inhibitors of Prostate Specific Antigen. Journal of Medicinal Chemistry, 2001. 44: 1491-1508.
12. Warren, P., et al., In Vitro Targeted Killing of Prostate Tumor Cells by a Synthetic Amoebapore Helix 3 Peptide Modified with Two γ-linked Glutamate Residues at the COOH Terminus. Cancer Research, 2001. 61: 6783-6787.
13. Fernandez, A.-M., et al., N-Succinyl-(β-alanyl-L-leucyl-L-alanyl-L-leucy)doxorubicin: An Extracellularly Tumor-Activated Prodrug Devoid of Intravenous Acute Toxicity. Journal of Medicinal Chemistry, 2001. 44: 3750-3753.
14. Trouet, A., et al., Extracellularly Tumor-activated Prodrugs for the Selective Chemotherapy of Cancer: Application to Doxorubicin and Preliminary in Vitro and in Vivo Studies. Cancer Research, 2001. 61: 2843-2846.
15. Dubois, V., et al., CPI-0004Na, a New Extracellularly Tumor-Activated Prodrug of Doxorubicin: In Vivo Toxicity, Activity, and Tissue Distribution Confirm Tumor Cell Selectivity. Cancer Research, 2002. 62: 2327-2331.
16. Berezov, A., et al., Disabling ErbB Receptors with Rationally Designed Exocyclic Mimetics of Antibodies: Structure-Function Analysis. Journal of Medicinal Chemistry, 2001. 44: 2565-2574.
17. Tsou, H.-R., et al., 6-Substituted-4-(3-bromophenylamino quinazolines as Putative Irreversible Inhibitors of the Epidermal Growth Factor Receptor (EGFR) and Human Epidermal Growth Factor Receptor (HER-2) Tyrosine Kinases with Enhanced Antitumor Activity. Journal of Medicinal Chemistry, 2001. 44: 2719-2734.
18. Cooke, S. P., et al., A Strategy for Antitumor Vascular Therapy by Targeting the Vascular Endothelial Growth Factor: Receptor Complex. Cancer Research, 2001. 61: 3653-3659.
19. Luwor, R. B., et al., Monoclonal Antibody 806 Inhibits the Growth of Tumor Xenografts Expressing Either the de2-7 or Amplified Epidermal Growth Factor Receptor (EGFR) but not Wild-Type EGFR. Cancer Research, 2001. 61: 5355-5361.
20. Foekens, J. A., et al., High Tumor Levels of Vascular Endothelial Growth Factor Predict Poor Response to Systemic Therapy in Advanced Breast Cancer. Cancer Research, 2001. 6,1: 5407-5414.
21. Kunkel, P., et al., Inhibition of Glioma Angiogenesis and Growth in Vivo by Systemic Treatment with a Monoclonal Antibody against Vascular Endothelial Growth Factor Receptor-2. Cancer Research, 2001. 61: 6624-6628.
22. Guo, P., et al., Vascular Endothelial Growth Factor Isoforms Display Distinct Activities in Promoting Tumor Angiogenesis at Different Anatomic Sites. Cancer Research, 2001. 61: 8569-8577.
23. Renhowe, P. A., Growth Factor Receptor Kinases in Cancer. Annual Reports in Medicinal Chemistry, 2001. 36: 109-118.
24. McVean, C. A., et al., Synthesis and In Vitro Potency of ανβ3 Receptor Antagonists: A Library Approach to RGD-Mimetics Incorporating Novel 3-Substituted β-alanines. 2001: Abstract Nat. ACS Meet.
25. Brashear, K. M., et al., Synthesis and In Vitro Potency of ανβ3 Receptor Antagonists: A Directed Approach to RGD-Mimetics Incorporating Novel 3-Substituted β-Alanines. 2001: Abstract Nat. ACS Meet.
26. Sulyok, G. A. G., et al., Solid-Phase Synthesis of a Nonpeptide RGD Mimetic Library: New Selective ανβ3 Integrin Antagonists. Journal of Medicinal Chemistry, 2001. 44: 1938-1950.
27. Reddy, J. A. and P. S. Low, Folate-Mediated Targeting of Therapeutic and Imaging Agents to Cancers. Critical Reviews in Therapeutic Drug Carrier Systems, 1998. 15: 587-627.
28. Lu, J. Y., et al., Folate-Targeted Enzyme Prodrug Cancer Therapy Utilizing Penicillin-V Amidase and a Doxonibicin Prodrug. Journal of Drug Targeting, 1999. 7: 43-53.
29. Ward, C. M., N. Acheson, and L. W. Seymour, Folic Acid Targeting of Protein Conjugates into Ascites Tumour Cells from Ovarian Cancer Patients. Journal of Drug Targeting, 2000. 8: 119-123.
30. Sudimack, J. and R. J. Lee, Targeted Drug Delivery via the Folate Receptor. Advanced Drug Delivery Reviews, 2000. 41: 147-162.
31. Wang, L., et al., Influence of Polyamine Architecture on the Transport and Topoisomerase II Inhibitory Properties of Polyamine DNA-Intercalator Conjugates. Journal of Medicinal Chemistry, 2001. 44: 3682-3691.
32. Roskelley, C. D., et al., Inhibition of Tumor Cell Invasion and Angiogenesis by Motuporamines. Cancer Research, 2001. 61: 6788-6794.
33. Phanstiel, O., et al., Polyamine-Assisted Delivery of Anticancer and Antimalarial Agents. 2002: Abstract 223rd ACS National Meeting, Orlando, Fla.
34. Paschke, R., et al., Highly Effective Cytotoxic Drugs, Synthesis and Activities of a Variety of Spacer-Linked Bile Acid Cisplatin and Carboplatin Derivatives. 2002: Abstract 223rd ACS National Meeting, Orlando, Fla.
35. Sweeney, C. J., et al., The Antiangiogenic Property of Docetaxel Is Synergistic with a Recombinant Humanized Monoclonal Antibody Against Vascular Endothelial Growth Factor or 2-Methoxyestradiol but Antagonized by Endothelial Growth Factors. Cancer Research, 2001. 61: 3369-3372.
36. Mentor-Marcel, R., et al., Genistein in the Diet Reduces the Incidence of Poorly Differentiated Prostatic Adenocarcinoma in Transgenic Mice (TRAMP). Cancer Research, 2001. 61: 6777-6782.
37. Ju, Y. H., et al., Dietary Genistein Negates the Inhibitory Effect of Tamoxifen on Growth of Estrogen-dependent Human Breast Cancer (MCF-7) Cells Implanted in Athymic Mice. Cancer Research, 2002. 62: 2474-2477.
38. Linja, M. J., et al., Amplification and Overexpression of Androgen Receptor Gene in Hormone-Refractory Prostate Cancer. Cancer Research, 2001. 61: 3550-3555.
39. Gregory, C. W., et al., A Mechanism for Androgen Receptor-mediated Prostate Cancer Recurrence after Androgen Deprivation Therapy. Cancer Research, 2001. 61: 4315-4319.
40. Manfredini, S., et al., Design, Synthesis and Activity of Ascorbic Acid Prodrugs or Nipecotic, Kynurenic and Diclophenamic Acids, Liable to Increase Neurotropic Activity. Journal of Medicinal Chemistry, 2002. 45: 559-562.
41. Klis, W. A., J. G. Sarver, and P. W. Erhardt, Mechanistic Considerations Pertaining To The Solvolysis Of Paclitaxel Analogs Bearing Ester Groups At The C2′ Position. Tetrahedron Letters, 2001. 42: 7747-7750.
42. Klis, W. A., Sarver, J. G., and Erhardt, P. W., Selective Conversion of 2′,7-Bis-Monochloroacetylpaclitaxel Analogs to 7-Monochloroacetyl Derivatives by Solvolysis In Methanol. Synthetic Communications, 2002. 32: 2711-2718.

We claim:

1. A method for treating cancer patients by administering at least one or more hybrid derivatives of paclitaxel that simultaneously display improved aqueous solubility, chemical stability under physiological conditions, and a decreased liability toward multi-drug resistance; said derivatives being deployed alone or in combination protocols with other chemotherapeutic agents.

2. The method of claim 1 wherein the hybrid derivatives contain at least one or more appendages attached to the 7-position of paclitaxel, the. 7-position of 10-deacetylpaclitaxel, the 10-position of 10-deacetylpaclitaxel, or the 10-position of 7-acyl-10-deacetylpaclitaxel; said acyl group including but not being limited to acetyl, chloroacetyl and methoxyacetyl.

3. The method of claim 2 wherein the attachments are via ester linkages which use the hydroxy group inherently present at the 7-position or the hydroxy group that becomes exposed at the 10-position after deacetylation of paclitaxel.

4. The method of claim 3 wherein the appendages are partially protected amino acids or are completely unprotected amino acids for which either appendage is attached via the amino acid's terminal or side-chain carboxylic acid moieties.

5. The method of claim 4 wherein the amino acid comprises [(CH₃)₃COCO]N-Asp, [φCO]N-Asp, Asp[CH(CH₃)₂], Asp-[CH₂φ], or Asp, and, wherein the amino acids utilize their side-chain, β-carboxylic acid moiety to form the ester linkage.

6. A method for treating cancer patients by administering at least one or more hybrid derivatives of paclitaxel that simultaneously display improved aqueous solubility, chemical stability under physiological conditions, a decreased liability toward multi-drug resistance, and enhanced selective toxicity toward cancer cells compared to normal cells.

7. The method of claim 6 wherein the appendages comprise adducts that are attached directly via an inherent carboxylic acid moiety or comprise adducts further connected to a linker molecule having a carboxylic acid that can serve as the attachment; said linker comprising a connecting chain between 1 to 10 carbons and also bearing additional chemical functionality that can increase aqueous solubility; said functionality including one or more combinations of an alcohol group, an amino group, or a carboxylic acid group.

8. The method of claim 7 wherein the adduct comprises a derivative of a small peptide; said peptide having two to ten amino acids in either a linear, branched or cyclic arrangement.

9. The method of claim 8 wherein the peptide comprises a Asn-Gly-Arg, [Acyl]N-Asn-Gly-Arg, Gly-Asn-Gly-Arg-Gly or Cys-Asn-Gly-Arg-Cys-Gly motif that preferentially distributes to the-neovascularization of a tumor.

10. The method of claim 8 wherein the peptide comprises a Arg-Gly-Asp, [Acyl]N-Arg-Gly-Asp, Arg-Gly-Asp-Ser, [Acyl]N-Arg-Gly-Asp-Ser, Arg-Gly-Asp-(D)-Phe-[N-Methyl]-Val or Cyclic(-Arg-Gly-Asp-(D)-Phe-[N-Methyl]-Val-) motif that preferentially distributes to integrin receptors over-expressed by cancer cells.

11. The method of claim 8 wherein the peptide comprises the γ-Glu-γ-Glu-NH₂ motif that associates with the PSMA enzyme produced by prostate cancer cells.

12. The method of claim 8 wherein the peptide comprises the Glutaryl-Hyp-Ala-Ser-Chg-Gln-Ser-Leu motif that associates with the PSA enzyme produced by prostate cancer cells.

13. The method of claim 8 wherein the peptide comprises the β-Ala-Leu-Ala-Leu or [HO₂C(CH₂)₂CO]N-β-Ala-Let-Ala-Leu motif that associates with a peptidase enzyme over-expressed by cancer cells.

14. The method of claim 6 wherein the adduct comprises a derivative of a 1,2,3-trisubstituted β-lactam system that inhibits the PSA enzyme produced by prostate cancer cells.

15. The method of claim 6 wherein the adduct comprises a derivative of a 4,6-disubstituted quinazoline system that associates with EGFR, HER-2 and ErbB pathways over-expressed within cancer cells.

16. The method of claim 6 wherein the adduct comprises a derivative of a 5,6,7,8-tetrahydro-1,8-naphthyyridin-2-yl system that preferentially distributes to integrin receptors over-expressed by cancer cells.

17. The method of claim 6 wherein the adduct comprises a derivative of folic acid that is able to use the folate transporter to enhance its uptake into cancer cells.

18. The method of claim 6 wherein the adduct comprises a derivative of spermine or of metuporamine C that is able to use the polyamino transporter to enhance its uptake into cancer cells or to decrease metastases by interrupting cancel cell invasion and motility.

19. The method of claim 6 wherein the adduct comprises a derivative of cholic acid that is able to use a cholate transporter to enhance its uptake into cancer cells.

20. The method of claim 6 wherein the adduct comprises a derivative of 2-methoxyestradiol or of genistein that associates with estrogen receptors over-expressed by cancer cells.

21. The method of claim 6 wherein the adduct comprises a derivative of testosterone that associates with androgen receptors over-expressed by cancer cells during early stage prostate cancer.

22. The method of claim 6 wherein the adduct comprises a derivative of ascorbic acid that is able to use a SVCT2 transporter to enhance its passage across a patient's blood-brain barrier so as to treat brain cancers.

23. A composition of matter comprising paclitaxel substituted with at least one or more polar appendages at either the 7- or 10-positions as defined by a formula 7-OR-10-OR′-paclitaxel wherein;
R is “Appendage-”, “Acyl-” or “H—”;
R′ is “Appendage-”, “Acetyl-” or “H—”;
“Appendage” is a polar adduct having a free carboxy-group so as to directly allow formation of an ester link to paclitaxel; is a polar adduct having at least one or more hydroxy- or amino-groups so that the adduct is attachable to a “Connecting chain” that then bears a free carboxy-group so as to allow formation of an ester link to paclitaxel; a non-polar adduct having a carboxy-, hydroxy- or amino-group so-that the non-polar adduct is attachable to a polar connecting chain that bears a free carboxy-group so as to allow formation of an ester link to paclitaxel;
“Acyl” is acetyl-, chloroacetyl- or methoxyacetyl-;
“Adduct” is at least one or more small peptide derivatives having from 2 to 10 amino acid units, small organic molecules having molecular weights less than 500 grams that are derivatives of the following templates: 1,2,3-trisubstituted β-lactam; 4,6-disubstituted quinazoline; 5,6,7,8-tetrahydro-1,8-naphthyridin-2-yl; folic acid; polyamine; metupuramine C; cholic acid; estrogen; phytoestrogen; androgen; or, ascorbic acid; and
“Connecting chain” is at least one of a non-polar alkyl or alkene straight or branched chain having 2 to 10 carbons and two carboxylic acid moieties, polar alkyl or alkene straight or branched chain having 2 to 10 carbons and three or more carboxy-, hydroxy- or amino-groups, or a non-polar or polar small peptide of 1 to 5 amino acids.

24. The composition of claim 23 wherein the appendage comprises a polar adduct having the formula [(CH₃)₃COCO]N-Asp, [φCH₂]N-Asp, Asp[CH(CH₃)₂], Asp-[CH₂φ], or Asp, all of which are directly linked to paclitaxel by their β-carboxylic acid moiety.

25. The composition of claim 23 wherein the appendage comprises a polar adduct having the formula Asn-Gly.-Arg, [Acyl]N-Asn-Gly-Arg, Gly-Asn-Gly-Arg-Gly, or c[-Cys-Asn-Gly-Arg-Cys-]Gly, all of which are directly linked to paclitaxel by their terminal carboxylic acid moiety.

26. The composition of claim 23 wherein the appendage comprises a polar adduct having the formula Arg-Gly-Asp, [Acyl]N-Arg-Gly-Asp, Arg-Gly-Asp-Ser, [Acyl]N-Arg-Gly-Asp-Ser, or c[-Arg-Gly-Asp-(D)-Phe[N—R′″]-Val-], all of which are directly linked to paclitaxel by, their terminal carboxylic acid moiety except for the cyclized motif which uses R′″═CH₂CH₂CO₂H to form said attachment.

27. The composition of claim 23 wherein the appendage comprises a polar adduct having the formula γ-Glu-γ-Glu-Gly directly linked to paclitaxel by the terminal carboxylic acid moiety.

28. The composition of claim 23 wherein the appendage comprises a polar adduct having a formula [Glutaryl]N-Hyp-Ala-Ser-Chg-Gln-Ser-Leu directly linked to paclitaxel by the terminal carboxylic acid moiety.

29. The composition of claim 23 wherein the appendage comprises a non-polar adduct having a formula β-Ala-Leu-Ala-Leu attached to a polar connecting chain by the terminal carboxylic acid moiety and wherein the polar connecting chain is then linked to paclitaxel by its own carboxylic acid moiety.

30. The composition of claim 23 wherein the appendage comprises a non-polar adduct having a formula of a 1,2,3-trisubstituted β-lactam system attached to a polar connecting chain by a carboxylic acid function and wherein the polar connecting chain is then linked to paclitaxel by its own carboxylic acid moiety.

31. The composition of claim 23 wherein the appendage comprises a non-polar adduct having a formula of a 4,6-disubstituted quinazoline system attached to a polar connecting chain by an amino function and wherein the polar connecting chain is then linked to paclitaxel by its own carboxylic acid moiety.

32. The composition of claim 23 wherein the appendage comprises a polar adduct having a formula of a 5,6,7,8-tetrahydro-1,8-naphthyridin-2-yl system whose side chain ends with an Asp directly linked to paclitaxel by the α-carboxylic acid moiety.

33. The composition of claim 23 wherein the appendage comprises a polar adduct having a formula of a folic acid derivative directly linked to paclitaxel by its terminal carboxylic acid moiety.

34. The composition of claim 23 wherein the appendage comprises a polar adduct having a formula of spermine or metuporamine C attached to a non-polar connecting chain by either a central or terminal amino function and wherein the non-polar connecting chain is then linked to paclitaxel by its own carboxylic acid moiety.

35. The composition of claim 23 wherein the appendage comprises a polar adduct having a formula of cholic acid, taurocholic acid or glycolic acid attached to a non-polar connecting chain by an amido function and wherein the non-polar connecting chain is then linked to paclitaxel by its own carboxylic acid moiety.

36. The composition of claim 23 wherein the appendage comprises a polar adduct having a formula of an aspartylcholic acid system directly linked to paclitaxel by its β-carboxylic acid moiety.

37. The composition of claim 23 wherein the appendage comprises a non-polar adduct having a formula of a 2-methoxyestradiol derivative attached to a polar connecting chain by an alcohol group and wherein the polar connecting chain is then linked to paclitaxel by its own carboxylic acid moiety.

38. The composition of claim 23 wherein the appendage comprises a moderately polar adduct having a formula of a genistein derivative attached to a non-polar or polar connecting chain by an alcohol group and wherein the connecting chain is then linked to paclitaxel by its own carboxylic acid moiety.

39. The composition of claim 23 wherein the appendage comprises a non-polar adduct having a formula of a testosterone derivative attached to a polar connecting chain by an alcohol group and wherein the connecting chain is then linked to paclitaxel by its own carboxylic acid moiety.

40. The composition of claim 23 wherein the appendage comprises a polar adduct having a formula of an ascorbic acid derivative that is attached to a non-polar connecting chain by an alcohol and wherein the non-polar connecting chain is then linked to paclitaxel by its own carboxylic acid moiety.
